(19) United States
(12) Patent Application Publication
(10) Pub. No.: US 2025/0283898 A1
(43) Pub. Date: Sep. 11, 2025

(54) ALPHA DEFENSIN ASSAY FOR DIAGNOSING NOSOCOMIAL VENTRICULITIS

(71) Applicant: UNIVERSITY OF MARYLAND, BALTIMORE, Baltimore, MD (US)

(72) Inventor: James DOUB, Perry Hall, MD (US)

(21) Appl. No.: 19/071,916

(22) Filed: Mar. 6, 2025

Related U.S. Application Data
(60) Provisional application No. 63/562,285, filed on Mar. 7, 2024.

(51) Int. Cl. G01N 33/68; A61K 31/407; A61K 31/546; A61K 38/14; A61K 45/06

(52) U.S. Cl. CPC G01N 33/6896; A61K 31/407; A61K 31/546; A61K 38/14; A61K 45/06; G01N 2333/4721; G01N 2800/28

(57) ABSTRACT

A method of using alpha defensins as a biomarker to aid in the diagnosis and treatment of ventriculitis. This biomarker can help improve diagnostic accuracy and decrease unwarranted empirical broad spectrum antibiotic use in suspected external ventricular drains (EVD)-associated ventriculitis.

### CROSS REFERENCE TO RELATED APPLICATIONS

This application is filed under the provisions of 35 U.S.C. § 111 (a) and claims priority to U.S. Provisional Patent Application No. 63/562,285 filed on Mar. 7, 2024 in the name of James DOUB, and entitled “Alpha Defensin Assay for Diagnosing Nosocomial Ventriculitis,” which is hereby incorporated by reference herein in its entirety.

### FIELD

The present invention relates to the use of α-defensins as a biomarker to aid in the diagnosis of ventriculitis, for example external ventricular drains (EVD)-associated ventriculitis.

### DESCRIPTION OF THE RELATED ART

Nosocomial ventriculitis is a life threating infection of the cerebral ventricles that is commonly associated with neurosurgical interventions, most notably with external ventricular drains (EVD) [Hasbun, 2021]. These are routinely used in neurological intensive care units to monitor and decant increased intracranial pressure [Tunkel et al., 2017]. Similar to indwelling catheters, EVDs are associated with increased risk of infections with prolonged durations of drain residence with an accepted overall incidence of approximately 10% [Id.].

Currently, the diagnosis of ventriculitis is conducted by evaluating symptomology and analyzing cerebral spinal fluid (CSF) for derangements of protein, glucose, cell count, gram stain and culture. Obstinately, EVD-associated ventriculitis is one of the hardest infectious disease syndromes to diagnosis. This stems from underlying neurological conditions such as intracerebral bleeding causing similar derangement of cerebral spinal fluid (CSF) white blood cells, protein and glucose to those seen with meningitis [Tunkel et al., 2017; Schade et al., 2007]. The result is that typical CSF parameters, for example as used in the diagnosis of meningitis, lack sensitivity and specificity in diagnosing nosocomial ventriculitis [Id.]. Moreover, in nosocomial ventriculitis, patients can have abnormal CSF parameters from underlying CNS pathologies complicating the diagnosis. To further complicate the diagnosis, indolent hard to culture bacteria, such as Cutibacterium acne and coagulase negative Staphylocccous spp., can be introduced into the ventricles by repeat manipulation or retrograde translocation [Tunkel et al., 2017; Bischoff et al., 2020]. Consequently, there is no gold standard in diagnosis and clinicians must rely on combined clinical symptoms and CSF parameters to make a probable diagnosis.

There is clearly a need for diagnostic tests and established standards to help clinicians diagnose nosocomial ventriculitis to thereby reduce morbidity and mortality and thwart the use and complications associated with the unwarranted administration of broad-spectrum antibiotics.

### SUMMARY

The present invention generally relates to using alpha defensins as a biomarker to aid in the diagnosis and treatment of ventriculitis.

In some aspects, a method of diagnosing and treating ventriculitis in a subject, said method comprising:
performing an assay to determine the amount of alpha defensins in a sample of cerebral spinal fluid (CSF) from the subject; and
administering a treatment to the subject if the amount of alpha defensins in the sample was determined by the assay to be greater than about 250 mg/L.

Different embodiments, features and advantages of the invention will be more fully apparent from the disclosure below and appended claims.

### BRIEF DESCRIPTION OF THE DRAWINGS

FIG. 1A shows the demographics and CSF analysis between the ventriculitis and the no ventriculitis cohort.

FIG. 1B shows the levels of α-defensin measured between the ventriculitis and the no ventriculitis cohort.

FIG. 2A. Evaluation of CSF alpha-defensin as a ventriculitis diagnostic. Violin graph showing a statistically significant difference (p<0.01) in concentrations of CSF alpha-defensins in CSF between the two cohorts. Dashed line represents cut off value of 220 mg/L.

FIG. 2B is an area under the curve graph showing AUC 0.9714, p<0.0001 (CI 0.9357 to 1.000).

FIG. 2C is a violin graph showing concentrations of alpha-defensins in CSF after seven days of antibiotics in which there was a significant difference (p<0.01) in concentrations compared to concentrations at time of initial diagnosis. Dashed line represents cutoff value 220 mg/L.

FIG. 2D is a violin graph showing stability of CSF alpha-defensin concentrations after four hours at room temperature in which there was no significant change in concentrations.

### DETAILED DESCRIPTION, AND PREFERRED EMBODIMENTS THEREOF

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “about,” when used in connection with one or more numbers or numerical ranges, should be understood to refer to all such numbers, including all numbers in a range and modifies that range by extending the boundaries slightly above and slightly below the numerical values set forth by, for example, +/−5%. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Alpha defensins” are small cationic peptides with 29-33 amino acids that are typically sequestered in neutrophil granules [Ganz T, 2003; Zhang H et al., 2001; Chairatana P et al., 2017]. Their structure comprises three beta strands forming a beta-sheet. These peptides are part of the innate antimicrobial molecules with broad antimicrobial activity to gram-positive and gram-negative bacteria, fungi and enveloped viruses [Ganz T, 2003]. They are released by activated neutrophils when they are exposed to microbes to disrupt microbial membrane integrity and function [Selsted M E, 1995]. Low concentrations of alpha defensins can be present in the serum of healthy patients, but concentrations increase significantly in infectious conditions causing sepsis [Zhang H et al., 2001; Chairatana P et al., 2017; Panyutich A V, 1993]. Importantly, high concentrations of these peptides are localized to areas where infections are present [Zhang H et al., 2001; Panyutich A V, 1993]. Towards that end, the present disclosure broadly relates to the use of alpha defensins as a diagnostic tool whereby increased concentrations of alpha defensins in specific locations substantially suggests an underlying infectious process.

An “amount” as used herein refers to a number e.g., where the number is a level, such as a position on a real or imaginary scale of amount or quantity, or a concentration, such as, for example, a relative amount of a given substance contained within a solution or in a particular volume of space, e.g., the amount of solute per unit volume of solution.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies, and functionally active epitope-binding fragments of any of the above. Antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody.”

“Determined by an assay” is used herein to refer to the determination of a reference level by any appropriate assay. The determination of a reference level may, in some embodiments, be achieved by an assay of the same type as the assay that is to be applied to the sample from the subject (for example, by an immunoassay). The determination of a reference level may, in some embodiments, be achieved by an assay of the same type and under the same assay conditions as the assay that is to be applied to the sample from the subject. As noted herein, this disclosure provides exemplary reference levels (e.g., calculated by comparing reference levels at different time points). It is well within the ordinary skill of one in the art to adapt the disclosure herein for other assays to obtain assay-specific reference levels for those other assays based on the description provided by this disclosure.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). In some embodiments, the alpha defensin reference levels referred to herein are from CSF. It is well within the ordinary skill of one in the art to adapt the disclosure herein for other immunoassays to obtain immunoassay-specific reference levels for those other immunoassays based on the description provided by this disclosure. Whereas the precise value of the reference level may vary between assays, the findings as described herein should be generally applicable and capable of being extrapolated to other assays.

A “sandwich immunoassay,” e.g., enzyme linked immunosorbent assay (ELISA), measures the amount of antigen between two layers of antibodies (i.e., at least one capture antibody) and a detection antibody (i.e., at least one detection antibody). The capture antibody and the detection antibody bind to different epitopes on the antigen, e.g., analyte of interest such as alpha defensins. Desirably, binding of the capture antibody to an epitope does not interfere with binding of the detection antibody to an epitope. Either monoclonal or polyclonal antibodies may be used as the capture and detection antibodies in the sandwich immunoassay. In some embodiments, antibodies are selected so that the one or more first antibodies brought into contact with a test sample suspected of containing analyte (e.g., alpha defensins) do not bind to all or part of an epitope recognized by the second or subsequent antibodies, thereby interfering with the ability of the one or more second detection antibodies to bind to the analyte.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. In some embodiments, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Treat,” “treating” or “treatment” are each used interchangeably herein to describe reversing, alleviating, or inhibiting the progress of a disease and/or injury, or one or more symptoms of such disease, to which such term applies. A treatment may be either performed in an acute or chronic way.

As discussed herein, current methods used to diagnose nosocomial ventriculitis are non-specific and/or not sensitive enough to be relied on. Disadvantageously, this has meant that either patients have been erroneously diagnosed and treated with prolonged courses of intravenous antibiotics when they don't need it, or other vulnerable patients that have underlying severe neurological conditions have been missed and are at risk for severe complications from not being adequately treated.

Presently, clinicians diagnose prosthetic joint infections, another difficult to diagnose infectious condition, using diagnostic tests involving the use of α-defensins [Doub, 2022]. Alpha defensins are an intrinsic antimicrobial peptide secreted by neutrophils when microbes are present [Ganz, 2003]. In prosthetic joint infections (PJI), this biomarker has been shown to have excellent sensitivity and specificity in determining prosthetic joint infection from synovial fluid samples [Bonanzinga et al., 2019]. Unexpectedly, α-defensins also have excellent sensitivity and specificity and can be used as a biomarker in diagnosing nosocomial ventriculitis.

Towards that end, in a first aspect, a method of diagnosing and treating ventriculitis in a subject is described, said method comprising:
performing an assay to determine the amount of alpha defensins in a sample of cerebral spinal fluid (CSF) from the subject; and
administering a treatment to the subject if the amount of alpha defensins in the sample was determined by the assay to be greater than about 250 mg/L.

In some embodiments, the subject is a human. In some embodiments, the subject has intracerebral hemorrhages. In some embodiments, the subject has intracerebral hemorrhages and has external ventricular drains. In some embodiments, the subject has culture positive ventriculitis. In some embodiments, the assay is an immunoassay. In some embodiments, the assay is an enzyme linked immunosorbent assay (ELISA). In some embodiments, the assay is a two-antibody ELISA using biotin-strepavidin-peroxidase detection. In some embodiments, the temperature of the assay performance is in a range from about 4° C. to about 25° C. In some embodiments, the temperature of the assay performance is room temperature. In some embodiments, the method further comprises obtaining the sample of CSF from the subject. In some embodiments, the ventriculitis is nosocomial ventriculitis.

In some other embodiments, the subject includes one of culture positive ventriculitis patients, or the subject has negative CSF cultures but has symptoms of ventriculitis as well as deranged CSF parameters with hypoglycorrhachia (less than 30 mg/dL or <50% of serum glucose) or lactate over 6 mmol/L and an infectious disease consultation deemed the subject to possibly have ventriculitis.

In some embodiments, the subject that had an amount of alpha defensins in the sample that was determined by the assay to be less than about 250 mg/L is not administered the treatment described herein.

In some embodiments, once diagnosed, the prescribed treatment entails antimicrobial treatment to prevent long term sequalae and reduce mortality. In some embodiments, the method of treatment comprises administration of systematic antimicrobial therapy and administration of intravenous vancomycin. In some embodiments, the method of treatment comprises administration of systematic antimicrobial therapy and administration of intravenous aminoglycosides selected from the group consisting of gentamicin, tobramycin, amikacin, neomycin, plazomicin, paromomycin, streptomycin, and any combination thereof. In some other embodiments, In some embodiments, the method of treatment comprises administration of systematic antimicrobial therapy, administration of intravenous vancomycin, and administration of intravenous aminoglycosides selected from the group consisting of gentamicin, tobramycin, amikacin, neomycin, plazomicin, paromomycin, streptomycin, and any combination thereof. In some embodiments, when administered, the dose of IV vancomycin is in a range from about 5 mg/day to about 30 mg/day. In some embodiments, when administered, IV vancomycin is administered for 1 day to 10 days. In some embodiments, when administered, the dose of IV aminoglycoside is in a range from about 5 mg/day to about 30 mg/day. In some embodiments, when administered, IV aminoglycoside is administered for 1 day to 10 days. In some embodiments, the systemic antimicrobial therapy comprises administration of a beta-lactam selected from cefepime, ceftazidime, or meropenem.

The features and advantages of the present invention will be understood more readily by reference to the examples discussed below, which are provided by way of illustration and are not intended to be limiting of the present invention.

### EXAMPLES

### Example 1

This study was approved by the University of Maryland Internal review board (HP-00103555). From May 1, 2022 through Dec. 30, 2022 the first ten patients admitted with intracerebral hemorrhages that were complicated by EVD-associated culture positive ventriculitis, had left-over CSF preserved by pipetting 400 μL of CSF into 2 mL cryovials (Corning) and freezing at −80° C. “Culture positive ventriculitis” was defined as having either 1) at least two separate positive CSF cultures or 2) one positive culture and a separate culture that was sterile but had a gram stain with the same bacteria visualized. During this same time period, ten individual intracerebral hemorrhage patients that did not have ventriculitis but had CSF obtained while an EVD was in place had their left-over CSF preserved as stated above. All patients included in this study had their demographics and CSF analysis recorded.

The twenty individual CSF samples were then evaluated for levels of α-defensins by use of a commercial two-antibody ELISA using biotin-strepavidin-peroxidase detection (R&D Systems). All twenty samples were run as one batch and tested in duplicates. In short, polystyrene plates (Maxisorb; Nunc) were coated with capture antibody in PBS overnight at 25° C. The plates were washed four times with 50 mM Tris, 0.2% Tween-20, pH 7.0-7.5 and then blocked for 90 minutes at 25° C. with assay buffer (PBS containing 4% BSA (Sigma)). Then 50 μl of CSF samples were added and incubated at 37° C. for 2 h. The plates were washed four times and 100 μl of biotinylated detecting antibody was added and incubated for 1 h at 25° C. After washing the plates, strepavidin-peroxidase polymer in casein buffer (RDI) was added and incubated at 25° C. for 30 min. The plate was washed another four times and 100 μl of commercially prepared substrate (TMB; Dako) was added and incubated at 25° C. for approximately 30 minutes. The reaction was stopped with 100 μl 2N HCl and the absorption (450 nm) was read on a microplate reader (Molecular Dynamics). A curve was fit to the standards using a computer program (SoftPro; Molecular Dynamics) and alpha defensin concentration in each sample was calculated from the standard curve equation. The same calibration curve was used for all the samples. Statistical evaluation was conducted using medcalc dot org (p value less than 0.05 was considered significant).

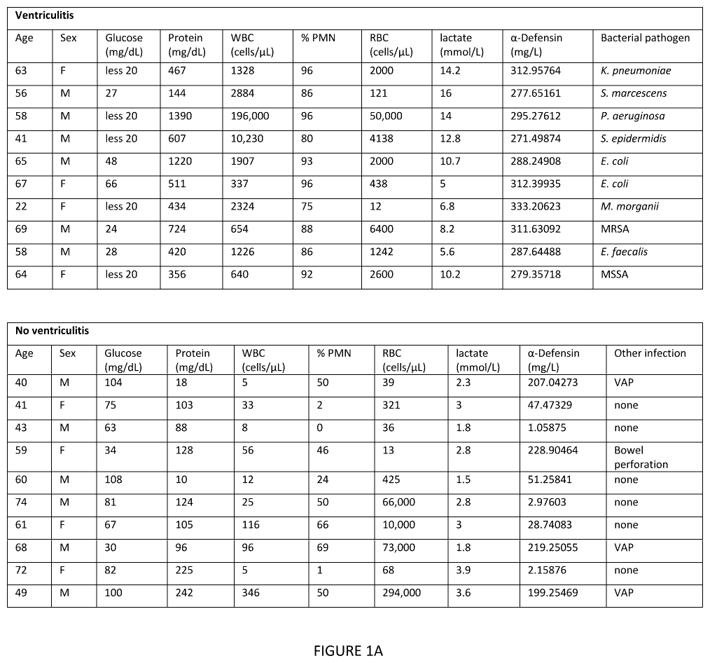

FIG. 1A shows the demographics and CSF analysis between the two cohorts. Notably the mean age was similar, 56.7 for the ventriculitis cohort and 56.3 for the cohort that did not have ventriculitis. In the cohort that had culture proven ventriculitis, 60% were composed of Gram-negative rods while 40% were composed of Gram-positive cocci. The cohort that did not have ventriculitis also had patients who had other infectious syndromes wherein three patients had ventilator associated pneumonia and one had a bowel perforation.

FIG. 1B shows the levels of α-defensin between the two cohorts. In the group with culture proven ventriculitis there was a statistically significant (P<0.0001) increased level of α-defensins in the CSF compared to those without ventriculitis. Notably, patients without ventriculitis that had other infectious syndromes had higher α-defensin levels compared to those that did not have other infectious conditions (circled data points). When this sub-cohort was compared to patients with known ventriculitis there was still a statistical difference (p<0.001).

There have been several studies looking at novel biomarkers to aid in the diagnosis of ventriculitis, but none have been shown to be particularly advantageous [Tunkel et al., 2017]. At the present time there is a paucity of research that has evaluated the use of α-defensin in central nervous system infections. In the few studies that have, α-defensin levels have been elevated in central nervous system infections [Fraisier et al., 2014; Maffei et al., 1999]. The experiments presented here reinforce that α-defensin can be used as a diagnostic in nosocomial ventriculitis in that there was a statistically significant difference in the amount of α-defensins in patients with known ventriculitis compared to those without ventriculitis.

While this example shows promise in using this biomarker in ventriculitis, there are no known cut-offs or defined assays to evaluate this biomarker in CSF samples. This is a known problem when evaluating novel infectious disease diagnostics in general [Patel et al., 2022]. In this example, all the patients with known ventriculitis had alpha defensin levels well over 250 mg/L and patients in the cohort without ventriculitis all had alpha defensin levels were well below 250 mg/L. This level is different than that used in chronic prosthetic joint infections, but these are drastically different infections with respect to acuteness and severity of infections [Doub, 2022; Bonanzinga et al., 2019]. Furthermore, a level of greater than 250 mg/L accommodates the fact that neurological intensive care patients are prone to have other infectious conditions that might lead to increased levels of alpha defensin in their CSF [Panyutich et al., 1993]. This example reinforces this biomarker cut-off given that four patients in the no ventriculitis cohort had other infectious conditions; when these non-ventriculitis CSF α-defensin levels were compared to patients with known ventriculitis there was still a statistical difference (p<0.0001). Therefore, this diagnostic can differentiate between infectious conditions which is important in the diagnosis of EVD-associated ventriculitis.

It is also important to note that the level of CSF α-defensins were not affected by large amounts of red blood cells in the CSF or with respect to different bacterial pathogens. This is advantageous because the majority of EVD-associated ventriculitis occurs in patients who have underlying intracerebral hemorrhages causing increased levels of red blood cells in CSF. Consequently, this example provides evidence that this biomarker has promise to help diagnose EVD-associated ventriculitis and not have results skewed by large amounts of red blood cells.

In conclusion, the findings evidence that α-defensins may be a biomarker to aid in the diagnosis of ventriculitis. This biomarker could be incorporated into ventriculitis diagnostic platforms to prevent morbidity and/or mortality as well as decrease unwarranted broad spectrum intravenous antibiotic use in suspected EVD-associated ventriculitis cases.

### Example 2

This study was approved by the University of Maryland Internal review board (HP-00103555). From 1/2019 through Jun. 30, 2024, patients that were older than 18 years old, had intracerebral hemorrhages requiring EVDs and underwent CSF analysis for normal clinical care had their left-over CSF obtained for investigation. CSF was obtained by pipetting at least 200 μL of left-over CSF into 2 mL cryovials and freezing these at −80° C. For the majority (n=75, 91%) of subjects, CSF was obtained from the EVD with the other seven samples being obtained through lumbar punctures.

Patients were deemed to have ventriculitis (n=41) if they had deranged CSF parameters, CSF cultures that grew microbes and were treated with antibiotic durations based on ventriculitis guidelines [Tunkel et al., 2017]. CSF parameters suggesting ventriculitis were pleocytosis with hypoglycorrhachia. Patients that had CSF obtained but had negative CSF cultures and were not treated with antibiotics were deemed to have true negative ventriculitis (n=41). Ten patients from the true ventriculitis cohort, that had repeat evaluation of CSF seven days after starting antibiotics, also had their repeat CSF preserved at −80° C. Furthermore, ten patients with ample residual CSF volumes had their CSF preserved at −80° C. and a portion of the same CSF was left at room temperature for four hours and then preserved at −80° C. Care was taken to only include patients with positive cultures, symptoms and whom infectious disease and neurocritical care clinicians deemed to have ventriculitis in the true ventriculitis cohort. As well, this biomarker was only assessed in patients with and without ventriculitis. Patients that had culture negative ventriculitis were not included, but this is the clinical cohort that would benefit most from this diagnostic.

The preserved CSF samples were then evaluated for levels of α-defensins by use of a two-antibody enzyme linked immunosorbent assay (ELISA) using biotin-strepavidin-peroxidase detection (R&D Systems) as described herein. Statistical evaluation was conducted using GraphPad prism (GraphPad software INC) with p value less than 0.05 being considered significant. The number of patient samples needed to produce a two-sided 95% confidence interval with a width of 20% when the AUC is 80% was 41 patients with culture proven ventriculitis and 41 patients with true negative ventriculitis.

TABLE 1
Demographics

| | Ventriculitis | | No ventriculitis | |
|---|---|---|---|---|
| | N = 41 | | N = 41 | |
| Age | 56.5 | (41-77) | 57.3 | (44-82) |
| Sex | 54% female | | 62% female | |
| Hypertension | 36 | (88%) | 35 | (85%) |
| Diabetes | 9 | (22%) | 12 | (29%) |
| Immunosuppressed | 4 | (10%) | 3 | (7%) |
| RBC in CSF | 41 | (100%) | 41 | (100%) |
| Pleocytosis in CSF ¹ | 41 | (100%) | 39 | (95%) |
| Hypoglycorrhachia in CSF ² | 41 | (100%) | 21 | (56%) |
| Alpha Defensin mg/L | 366.9 | (153.8-790.9) | 57.6 | (0.4-417) |
| mean (range) | | | | |

¹ Pleocytosis as defined as greater than 5 cells/mm³ after correcting for red blood cells
² Hypoglycorrhachia as defined as CSF to serum glucose ratio ≤0.5

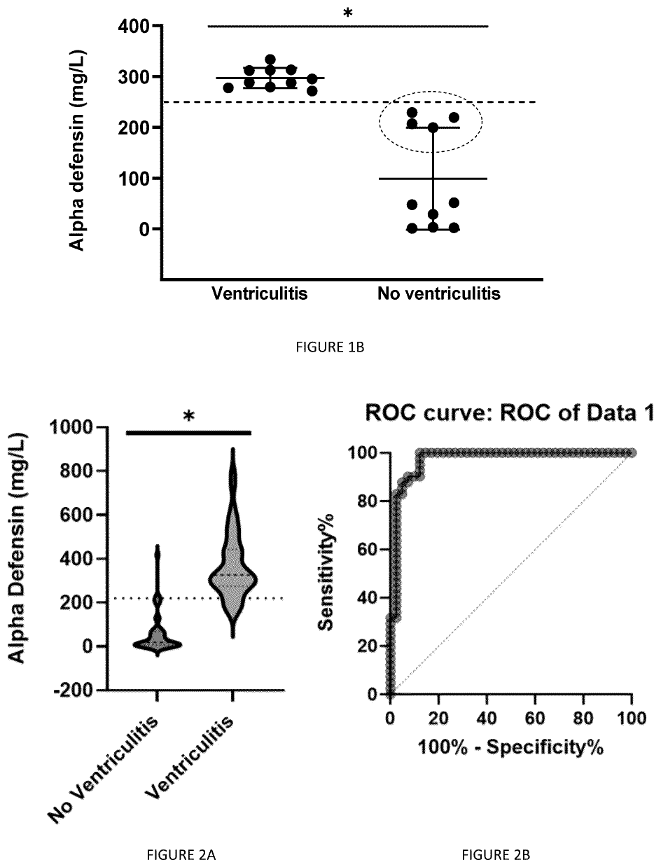

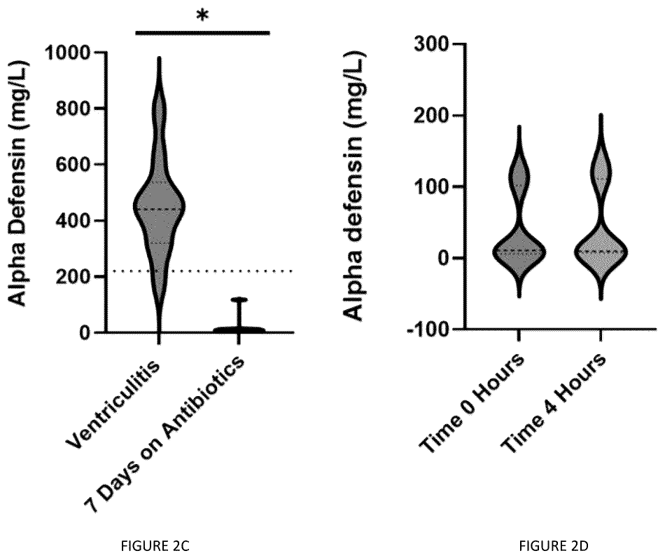

Table 1 shows the demographics of the cohorts. To note n=39 (95%) in the true negative cohort had pleocytosis as defined as greater than 5 white cells per mm³. When the level of CSF alpha-defensins in the ventriculitis cohort (M 366.9 mg/L, SD 148.6) was compared to the no ventriculitis cohort (M 57.6 mg/L, SD 86.1), there was a statistically significant difference (p<0.01) (FIG. 2A). Most patients with no ventriculitis had low levels of alpha-defensins (less than 220 mg/L) in their CSF. There were two patients in the non-ventriculitis cohort that had alpha-defensin level over 220 mg/L, but these patients were also suffering from persistent bacteremia despite having CSF parameters that did not suggest that they had ventriculitis. Moreover, the receiver operator curve (ROC) (FIG. 2B) shows an area under curve (AUC) of 0.9714, p<0.0001 (95% CI 0.9357 to 1.000). When using the cutoff value of 220 mg/L there was correlated sensitivity of 85.37% (CI 71.56% to 93.12%) and specificity of 95.12% (CI 83.86% to 99.13%).

When patients who suffered from ventriculitis had the concentration of CSF alpha-defensin evaluated before (M 452 mg/L, SD 170.9), compared after seven days of antibiotics (M 20.67 mg/L, SD 34.1), there was a statistically significant (p<0.01) decrease in this biomarker in the individuals CSF (FIG. 2C). On the other hand, when this biomarker was evaluated for stability at room temperature, there was no statistically significant change in the amount of alpha-defensins in the CSF at time 0 (M 39.65 mg/L, SD 50.67) compared to four hours later (M 42.42 mg/L, SD 54.73) (FIG. 2D).

Overall, this example suggests that alpha-defensin can be a reliable biomarker for nosocomial ventriculitis in that there was a statistically significant difference (p<0.01) in alpha-defensin levels between patients with ventriculitis compared to those without (FIG. 2A). In patients without ventriculitis but whom had pleocytosis, their innate immune cells did not secrete high levels of alpha-defensins, which support the theory that the presence of microbes are needed to stimulate innate immune cells to secrete alpha-defensins in the CSF. Correlated with this, in patients without ventriculitis, the levels of alpha-defensins were typically <220 mg/L. Suggesting that when no CNS infection is present, the levels of alpha-defensins in the CSF are typically low. Yet, when patients suffer from severe systemic infections, such as persistent bacteremia, elevated levels of alpha-defensins in CSF can also occur given alpha-defensins can pass through the blood brain barrier. However, evaluating patients for alternative infectious processes are important and are routinely conducted by clinicians when evaluating ventriculitis [Tunkel et al., 2017].

Nonetheless, the use of alpha-defensins for diagnosing of nosocomial ventriculitis had a ROC AUC of 0.9714 which demonstrates excellent performance of CSF alpha-defensin as a biomarker for ventriculitis with regards to both its specificity and sensitivity (FIG. 2B). When using a cutoff of 220 mg/L, there was excellent specificity (95.12%) and sensitivity (85.3%). These findings are important because at the present time numerous CSF markers such as lactate, glucose and others lack significant sensitivity and specificity for diagnosing ventriculitis [Tunkel et al., 2017]. Thus, the findings here support further development of this biomarker in the diagnosis of nosocomial ventriculitis.

While proving the successful performance of this diagnostic is vital, ensuring that this biomarker does not have persistent elevated levels in the CSF for prolonged periods of time after treatment with antibiotics is also important. In this study, a subset of patients with ventriculitis had repeat CSF alpha-defensin measured after seven days of antibiotic therapy. All patients of this subset had a statistically significant (p<0.01) decrease in alpha-defensin levels after treatment with antibiotics. Importantly, these results demonstrate that after elimination of microbes, innate immune cells stop secreting this biomarker and elevated levels of this biomarker are thus cleared from the CSF. Further, this study also showed that alpha-defensins are stable at room temperature for four hours. As a result, left-over CSF, could theoretically be used to determine alpha-defensin concentrations to aid in diagnosing ventriculitis.

In conclusion, CSF alpha-defensin has excellent sensitivity and specificity in diagnosing nosocomial ventriculitis and is stable at room temperature for at least four hours.

Although the invention has been disclosed herein in details with reference to various embodiments and features, it will be appreciated by a person with ordinary skill in the art that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Bischoff, P., Schröder, C., Gastmeier, P., Geffers, C. (2020). Surveillance of external ventricular drainage-associated meningitis and ventriculitis in German intensive care units. Infection Control & Hospital Epidemiology, 41 (4), 452-457. doi: 10.1017/ice.2019.367.
Bonanzinga T, Ferrari M C, Tanzi G, Vandenbulcke F, Zahar A, Marcacci M. The role of alpha defensin in prosthetic joint infection (PJI) diagnosis: a literature review. EFORT Open Rev. 2019 Jan. 23; 4 (1): 10-13. doi: 10.1302/2058-5241.4.180029.
Chairatana P, Nolan E M. Human α-Defensin 6: A Small Peptide That Self-Assembles and Protects the Host by Entangling Microbes. Acc Chem Res. 2017; 50 (4): 960-967. doi: 10.1021/acs.accounts.6b00653
Doub J B. Use of α-Defensins to Help Diagnose Nosocomial Ventriculitis [published online ahead of print, 2022 Nov. 30]. Neurocrit Care. 2022; 1-4. doi: 10.1007/s12028-022-01643-8.
Fraisier C, Papa A, Granjeaud S, et al. Cerebrospinal fluid biomarker candidates associated with human WNV neuroinvasive disease. PLOS One. 2014; 9 (4): e93637. Published 2014 Apr. 2.
Ganz T. Defensins: antimicrobial peptides of innate immunity. Nat Rev Immunol 2003; 3:710-720.
Hasbun R. Healthcare-associated ventriculitis: current and emerging diagnostic and treatment strategies. Expert Rev Anti Infect Ther. 2021; 19 (8): 993-999.
Maffei F A, Heine R P, Whalen M J, Mortimer L F, Carcillo J A. Levels of antimicrobial molecules defensin and lactoferrin are elevated in the cerebrospinal fluid of children with meningitis. Pediatrics. 1999; 103 (5 Pt 1): 987-992.
Panyutich A V, Panyutich E A, Krapivin V A, Baturevich E A, Ganz T. Plasma defensin concentrations are elevated in patients with sepsis or bacterial meningitis. J Lab Clin Med 1993; 122:202-207.
Patel R, Tsalik E L, Evans S, Fowler V G, Doernberg S B; Antibacterial Resistance Leadership Group. Clinically Adjudicated Reference Standards for Evaluation of Infectious Diseases Diagnostics [published online ahead of print, 2022 Oct. 20]. Clin Infect Dis. 2022; ciac829. doi: 10.1093/cid/ciac829.
Schade R P, Schinkel J, Roelandse F W, et al. Lack of value of routine analysis of cerebrospinal fluid for prediction and diagnosis of external drainage-related bacterial meningitis. J Neurosurg. 2007 May; 106 (5): 941.
Selsted M E, White S H, Wimley W C. Structure, function, and membrane integration of defensins. Curr. Opin. Struct. Biol. 1995: 5 (4): 521-527.
Tunkel A R, Hasbun R, Bhimraj A, et al. 2017 Infectious Diseases Society of America's Clinical Practice Guidelines for Healthcare-Associated Ventriculitis and Meningitis. Clin Infect Dis. 2017; 64 (6): e34-e65.
Zhang H, Porro G, Orzech N, Mullen B, Liu M, Slutsky A S. Neutrophil defensins mediate acute inflammatory response and lung function in a dose-dependent fashion. Am J Physiol Lung Cell Mol Physiol. 2001; 280: L947-L954.

What is claimed is:

1. A method of diagnosing and treating ventriculitis in a subject, said method comprising:
performing an assay to determine the amount of alpha defensins in a sample of cerebral spinal fluid (CSF) from the subject; and
administering a treatment to the subject if the amount of alpha defensins in the sample was determined by the assay to be greater than about 250 mg/L.

2. The method of claim 1, the subject is a human.

3. The method of claim 1, wherein the subject has intracerebral hemorrhages.

4. The method of claim 1, wherein the subject has intracerebral hemorrhages and has external ventricular drains.

5. The method of claim 1, wherein the subject has culture positive ventriculitis.

6. The method of claim 1, wherein the ventriculitis is nosocomial ventriculitis.

7. The method of claim 1, wherein the assay is an immunoassay.

8. The method of claim 7, wherein the assay is an enzyme linked immunosorbent assay (ELISA).

9. The method of claim 8, wherein the assay is a two-antibody ELISA using biotin-strepavidin-peroxidase detection.

10. The method of claim 1, wherein a temperature of assay performance is in a range from about 4° C. to about 25° C.

11. The method of claim 1, further comprising obtaining the sample of CSF from the subject.

12. The method of claim 1, wherein the treatment comprises antimicrobial treatment.

13. The method of claim 12, wherein the treatment comprises administration of systematic antimicrobial therapy and administration of intravenous vancomycin.

14. The method of claim 12, wherein the treatment comprises administration of systematic antimicrobial therapy and administration of intravenous aminoglycosides selected from the group consisting of gentamicin, tobramycin, amikacin, neomycin, plazomicin, paromomycin, streptomycin, and any combination thereof.

15. The method of claim 12, wherein the treatment comprises administration of systematic antimicrobial therapy, administration of intravenous vancomycin, and administration of intravenous aminoglycosides selected from the group consisting of gentamicin, tobramycin, amikacin, neomycin, plazomicin, paromomycin, streptomycin, and any combination thereof.

16. The method of claim 13, wherein the dose of intravenous vancomycin is in a range from about 5 mg/day to about 30 mg/day.

17. The method of claim 16, wherein intravenous vancomycin is administered for 1 day to 10 days.

18. The method of claim 14, wherein the dose of intravenous aminoglycoside is in a range from about 5 mg/day to about 30 mg/day.

19. The method of claim 18, wherein intravenous aminoglycoside is administered for 1 day to 10 days.

20. The method of claim 13, wherein the systemic antimicrobial therapy comprises administration of a beta-lactam selected from cefepime, ceftazidime, or meropenem.
